(19) United States
(12) Patent Application Publication
(10) Pub. No.: US 2015/0203812 A1
(43) Pub. Date: Jul. 23, 2015

(54) Inhibitors of Voltage Gated Ion Channels

(71) Applicant: Francesco Tombola, Irvine, CA (US)

(72) Inventor: Francesco Tombola, Irvine, CA (US)

(21) Appl. No.: 14/159,416

(22) Filed: Jan. 20, 2014

(51) Int. Cl. C12N 5/071

(52) U.S. Cl. CPC C12N 5/0602; C12N 2500/46

(57) ABSTRACT

Certain embodiments of the present invention provide compounds that comprise at least one guanidine moiety and have a binding affinity for the voltage sensing domain (VSD) of a voltage-gated ion channel, such as the Hv1 voltage-gated proton channel. In some embodiments, the compounds of the present invention that have as VSD binding affinity are further characterized by having a property of inhibiting ion transport by the voltage-gated ion channel comprising the VSD, such as inhibiting proton transport by Hv1. In some embodiments, the compounds according to the invention comprise at least one guanidine moiety in a five-membered aromatic ring.

### PRIORITY DATA

The present application claims the benefit of U.S. Provisional Patent Application Ser. No. 61/754,906, which is hereby incorporated by reference in its entirety and which was filed Jan. 21, 2013.

This invention was made in part with United States Government support under Grant No. R01GM098973, awarded by the National Institutes of Health. The U.S. Government has certain rights in this invention.

### FIELD OF THE INVENTIONS

Embodiments of the present invention relate to voltage sensing domains (VSD) of voltage-gated ion channels, engineered derivatives thereof, and mutants thereof. In some embodiments, such VSDs are of the Hv1 voltage-gated proton channel. Some embodiments of the present invention relate to modifiers of Hv1 proton transport activity. Some embodiments relate to small molecule modifiers of voltage-gated ion channel transport activity, compositions thereof, and uses thereof. Some embodiments relate to biologic molecule modifiers of voltage-gated ion channel transport activity, compositions thereof, and uses thereof. Some embodiments relate to combinations of one or more small molecule and/or one or more biologic molecule modifiers of voltage-gated ion channel transport activity, compositions thereof, and uses thereof.

### BACKGROUND OF THE INVENTIONS

Voltage gated ion channels, such as potassium, sodium, calcium, and proton voltage gated channels, comprise evolutionarily conserved VSDs characterized by four membrane-spanning segments (S1 through S4) operative to detect changes in cell membrane potential. Voltage-gated sodium, potassium, and calcium channels further comprise a pore domain (PD) operative to provide selective ion permeation. A gate located on the intracellular side of the PD (i.e., the activation gate) opens and closes the ion channel of a PD as a function of direct, membrane-potential-dependent interaction with VSDs. A large number of small molecules are known to inhibit PD ion transport by acting as open channel blockers. Such small molecule inhibitors have been shown to bind and block ion permeation channels of a PD only when its gate is open.

The wild-type Hv1 voltage gated proton channel (also known as HVCN1 or VSOP) forms a homodimer that comprises two VSDs and lacks a PD. Previously, the location of the activation gate in the Hv1 VSD was unknown and open channel blockers for the Hv1 VSD were also unknown.

The Hv1 voltage-gated proton channel has been shown to play important roles in proton extrusion, pH homeostasis, and production of reactive oxygen species in a variety of cell types. Hv1 has been found to be highly expressed in breast cancer cells; and its knockdown by RNA interference has been shown to strongly reduce cell proliferation and invasiveness. Hv1 activity has also been shown to be involved in NOX-mediated neuronal death during cerebral ischemia; and mice lacking Hv1 activity have been shown to be protected from brain damage after stroke.

### SUMMARY OF THE INVENTIONS

Certain embodiments of the present invention provide methods of inhibiting ion transport by a voltage-gated ion channel in a subject in need thereof. In some embodiments, the ion is a proton and the voltage-gated ion channel is a voltage gated proton channel, such as Hv1. In some embodiments, the subject comprises a plurality of cells that express the voltage-gated proton channel, and the voltage-gated proton channel comprises a VSD. The methods involve administering to the subject a therapeutically effective amount of a compound that: (i) is capable of binding to the VSD; (ii) is capable of inhibiting proton transport by the voltage-gated proton channel, and (iii) comprises a chemical structure according to formula I:

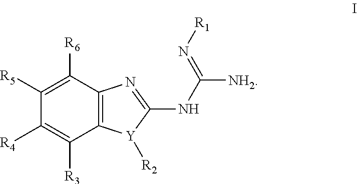

In formula I, Y is either N or S; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is S.

In some embodiments, the compound according to formula I comprises at least one member selected from the group consisting of a 2-aminobenzimidazole, a 2-guanidinobenzimidazole, a 2-guanidino-4-methylquinazoline, 1-(1,3-ben-zothiazol-2-yl)guanidine, and a 5-chloro-2-guanidinobenzimidazole.

In some embodiments, the subject is a mammal. In some embodiments, the subject is a human.

Certain embodiments of the present invention provide methods of inhibiting ion transport by a voltage-gated ion channel in a subject in need thereof. In some embodiments, the ion is a proton and the voltage-gated ion channel is a voltage gated proton channel, such as Hv1. In some embodiments, the subject comprises a plurality of cells that express the voltage-gated proton channel, and the voltage-gated proton channel comprises a VSD. The methods involve administering to the subject a therapeutically effective amount of a compound that: (i) is capable of binding to the VSD; (ii) is capable of inhibiting proton transport by the voltage-gated proton channel, and (iii) comprises a chemical structure according to formula II:

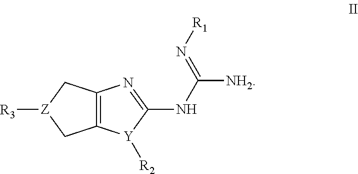

In formula II, Z is either C or N; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is S.

In some embodiments, the subject is a mammal. In some embodiments, the subject is a human.

Certain embodiments of the present invention provide methods of inhibiting ion transport by a voltage-gated ion channel in a subject in need thereof. In some embodiments, the ion is a proton and the voltage-gated ion channel is a voltage gated proton channel, such as Hv1. In some embodiments, the subject comprises a plurality of cells that express the voltage-gated proton channel, and the voltage-gated proton channel comprises a VSD. The methods involve administering to the subject a therapeutically effective amount of a compound that: (i) is capable of binding to the VSD; (ii) is capable of inhibiting proton transport by the voltage-gated proton channel, and (iii) comprises a chemical structure according to formula III:

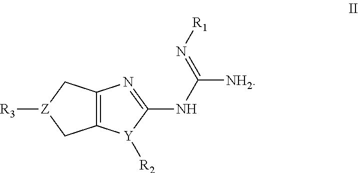

In formula III, Y is either N or S; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is S.

In some embodiments, the subject is a mammal. In some embodiments, the subject is a human.

Certain embodiments of the present invention provide methods of inhibiting at least one of cancer cell proliferation, migration, and invasion in a subject in need thereof. The methods involve contacting a plurality of cancer cells in the subject with an amount of a compound sufficient to inhibit at least one of cancer cell proliferation, migration, and invasion. At least a portion of the plurality of cancer cells express a voltage-gated ion channel characterized by having a VSD and an ion transport activity. In some embodiments, the voltage-gated ion channel is a proton channel, such as Hv1, and the ion transport activity is a proton transport activity. And the compound is: (i) capable of binding to the VSD; (ii) capable of inhibiting the proton transport activity, and (iii) comprises a chemical structure according to formula I:

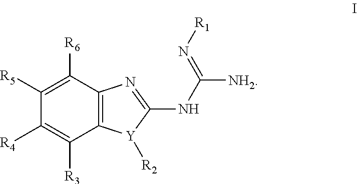

In formula I, Y is either N or S; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is S.

In some embodiments, the compound according to formula I comprises at least one member selected from the group consisting of a 2-aminobenzimidazole, a 2-guanidinobenzimidazole, a 2-guanidino-4-methylquinazoline, 1-(1,3-ben-zothiazol-2-yl)guanidine, and a 5-chloro-2-guanidinobenzimidazole.

In some embodiments, the subject is a mammal. In some embodiments, the subject is a human.

Certain embodiments of the present invention provide methods of inhibiting at least one of cancer cell proliferation, migration, and invasion in a subject in need thereof. The methods involve contacting a plurality of cancer cells in the subject with an amount of a compound sufficient to inhibit at least one of cancer cell proliferation, migration, and invasion. At least a portion of the plurality of cancer cells express a voltage-gated ion channel characterized by having a VSD and an ion transport activity. In some embodiments, the voltage-gated ion channel is a proton channel, such as Hv1, and the ion transport activity is a proton transport activity. And the compound is: (i) capable of binding to the VSD; (ii) capable of inhibiting the proton transport activity, and (iii) comprises a chemical structure according to formula II:

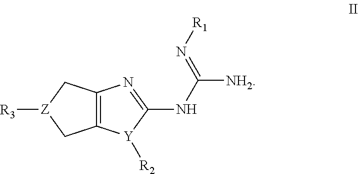

In formula II, Z is either C or N; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is S.

In some embodiments, the subject is a mammal. In some embodiments, the subject is a human.

Certain embodiments of the present invention provide methods of inhibiting at least one of cancer cell proliferation, migration, and invasion in a subject in need thereof. The methods involve contacting a plurality of cancer cells in the subject with an amount of a compound sufficient to inhibit at least one of cancer cell proliferation, migration, and invasion. At least a portion of the plurality of cancer cells express a voltage-gated ion channel characterized by having a VSD and an ion transport activity. In some embodiments, the voltage-gated ion channel is a proton channel, such as Hv1, and the ion transport activity is a proton transport activity. And the compound is: (i) capable of binding to the VSD; (ii) capable of inhibiting the proton transport activity, and (iii) comprises a chemical structure according to formula II:

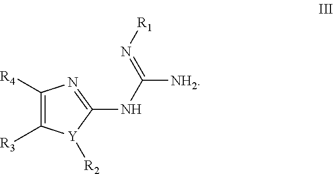

In formula III, Y is either N or S; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is S.

In some embodiments, the subject is a mammal. In some embodiments, the subject is a human.

Certain embodiments of the present invention provide methods of inhibiting production of reactive oxygen species in a subject in need thereof. The methods involve contacting a plurality of cells in the subject with an amount of a compound sufficient to inhibit production of reactive oxygen species. At least a portion of the cells express a voltage-gated ion channel characterized by having a VSD and an ion transport activity. In some embodiments, the voltage-gated ion channel is a proton channel, such as Hv1, and the ion transport activity is a proton transport activity. And the compound is: (i) capable of binding to the VSD; (ii) capable of inhibiting the proton transport activity, and (iii) comprises a chemical structure according to formula I:

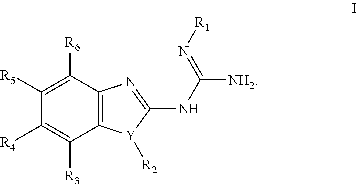

In formula I, Y is either N or S; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is S.

In some embodiments, the compound according to formula I comprises at least one member selected from the group consisting of a 2-aminobenzimidazole, a 2-guanidinobenzimidazole, a 2-guanidino-4-methylquinazoline, 1-(1,3-ben-zothiazol-2-yl)guanidine, and a 5-chloro-2-guanidinobenzimidazole.

In some embodiments, the subject is a mammal. In some embodiments, the subject is a human.

In some embodiments, the plurality of cells comprises at least one of a brain microglial cell, a blood cell, a macrophage, and a neutrophil.

In some embodiments, the reactive oxygen species comprises at least one of a superoxide hydrogen peroxide and peroxynitrite. In some embodiments, the production of reactive oxygen species is mediated by an NADPH oxidase.

Certain embodiments of the present invention provide methods of inhibiting production of reactive oxygen species in a subject in need thereof. The methods involve contacting a plurality of cells in the subject with an amount of a compound sufficient to inhibit production of reactive oxygen species. At least a portion of the cells express a voltage-gated ion channel characterized by having a VSD and an ion transport activity. In some embodiments, the voltage-gated ion channel is a proton channel, such as Hv1, and the ion transport activity is a proton transport activity. And the compound is: (i) capable of binding to the VSD; (ii) capable of inhibiting the proton transport activity, and (iii) comprises a chemical structure according to formula II:

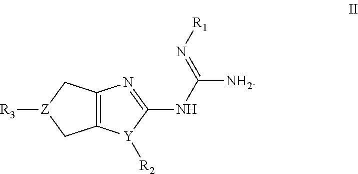

In formula II, Z is either C or N; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is S.

In some embodiments, the subject is a mammal. In some embodiments, the subject is a human.

In some embodiments, the plurality of cells comprises at least one of a brain microglial cell, a blood cell, a macrophage, and a neutrophil.

In some embodiments, the reactive oxygen species comprises at least one of a superoxide hydrogen peroxide and peroxynitrite. In some embodiments, the production of reactive oxygen species is mediated by an NADPH oxidase.

Certain embodiments of the present invention provide methods of inhibiting production of reactive oxygen species in a subject in need thereof. The methods involve contacting a plurality of cells in the subject with an amount of a compound sufficient to inhibit production of reactive oxygen species. At least a portion of the cells express a voltage-gated ion channel characterized by having a VSD and an ion transport activity. In some embodiments, the voltage-gated ion channel is a proton channel, such as Hv1, and the ion transport activity is a proton transport activity. And the compound is: (i) capable of binding to the VSD; (ii) capable of inhibiting the proton transport activity, and (iii) comprises a chemical structure according to formula III:

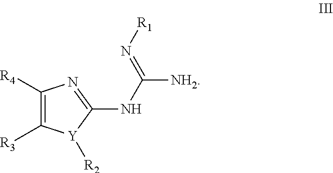

In formula III, Y is either N or S; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is S.

In some embodiments, the subject is a mammal. In some embodiments, the subject is a human.

In some embodiments, the plurality of cells comprises at least one of a brain microglial cell, a blood cell, a macrophage, and a neutrophil.

In some embodiments, the reactive oxygen species comprises at least one of a superoxide hydrogen peroxide and peroxynitrite. In some embodiments, the production of reactive oxygen species is mediated by an NADPH oxidase.

### BRIEF DESCRIPTION OF THE DRAWINGS

FIG. 1A is a schematic illustration of a potassium voltage gated channel (Kv) inserted in a cell membrane that separates extracellular (o) and intracellular (i) spaces. The Kv VSD comprises cell membrane spanning domains S1-S4 and the Kv PD comprises membrane spanning domains S5 and S6. The N-terminus of Kv is labeled N and the C-terminus of Kv is labeled C. FIG. 1B is a schematic illustration of a proton voltage gated channel (Hv1) inserted in a cell membrane that separates extracellular (o) and intracellular (i) spaces. The Hv1 VSD comprises cell membrane spanning domains S1-S4; and Hv1 lacks a PD. The coiled coil domain of Hv1 is labeled CCD. The N-terminus of Hv1 is labeled N and the C-terminus of Hv1 is labeled C.

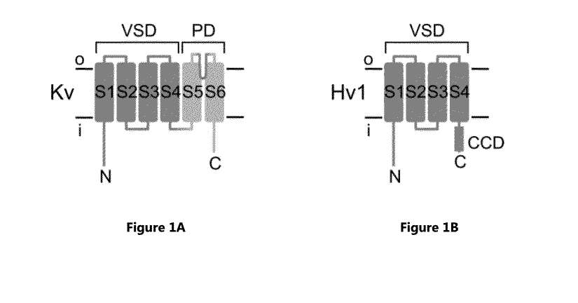

FIG. 2 shows the chemical structure of guanidine and related compounds. [1] Guanidine, [2] amino-guanidine, [3] 2-aminoimidazole, [4] 2-aminopyrimidine, [5] agmatine, [6] 2-aminobenzimidazole, [7] 2-guanidinobenzimidazole, [8] 1,1-dimethylbiguanide, [9] 2-guanidino-4-methylquinazoline, [10] N-(guanidino-iminomethyl)-N-phenylacetamide, [11] 1-(1,3-benzoxazol-2-yl)guanidine, [12] 1-(1,3-benzothiazol-2-yl)guanidine, [13] 5-chloro-2-guanidinobenzimidazole, [14] ethyl 2-[[amino(imino)methyl]amino]-5-methyl-1,3-thiazole-4-carboxylate, [15] ethyl 2-[[amino(imino) methyl]amino]-4-methyl-1,3-thiazole-5-carboxylate, [16] ethyl (2-[[amino(imino)methyl]amino1-4-methyl-1,3-thiazol-5-yl)acetate, [17] 3-([2-(diaminomethyleneamino) thiazol-4-yllmethylthio)-N′-sulfamoylpropanimidamide, [18] isopropyl 6-(guanidinoimino)-6-phenylhexanoate, [19] 1-[(4-fluorophenyl)methyl]-N-[1-[2-(4-methoxyphenyl)ethyl]-4-piperidylibenzoimidazol-2-amine, [20] 5-nitro-2-guanidinobenzimidazole, and [21] N(3)-(4-chlorophenyl)-4H-1,2,4-triazole-3,5-diamine. Guanidine moieties are highlighted in gray in compounds 1-7. The parts of compound 7 that are conserved in compounds 8-12 are also highlighted in gray.

FIG. 3 is a graph of the average Hv1 proton transport inhibition produced in the Xenopus oocyte patch clamp assay described in Example 3 by compounds 1-12 illustrated in FIG. 2 added intracellularly at 200 μM.

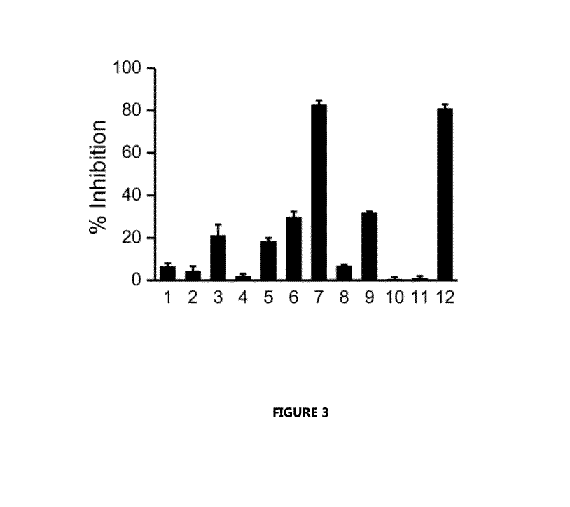

FIG. 4A is a chart of currents measured in an inside-out patch from a Xenopus oocyte expressing WT human Hv1 before (gray trace) and after addition of 200 μM compound 7 in FIG. 2 (black trace). Light gray trace (overlapping gray trace) is the current measured after washout of compound 7. Currents were activated by depolarizations to +120 mV from a holding potential of −80 mV. pH_{IN}=pH_{OUT}=6.0. FIG. 4B shows currents from native proton channels expressed in a mouse BV-2 microglial cell (left) and a human THP-1 monocyte/macrophage cell (right) before (gray trace) and after (black trace) addition of 200 μM compound 13 (as shown in FIG. 2) in the extracellular solution. Currents were measured in whole-cell patch configuration with pH_{IN}=6.0 and pH_{OUT}=7.5. The membrane was depolarized to +120 mV from a holding potential of −80 mV.

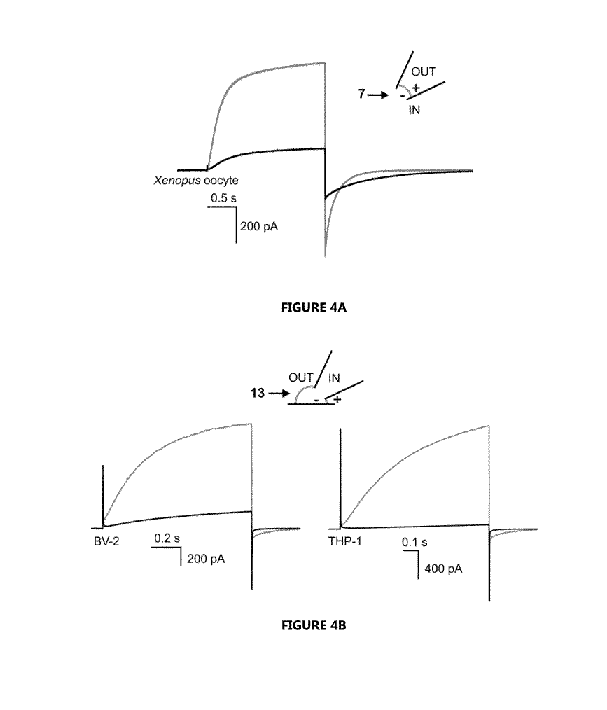

FIG. 5A shows photographs of wound healing assays conducted as described in Example 4 and with a DMSO vehicle and compounds 11, 7, and 13 illustrated in FIG. 2 on breast cancer cell line MB-231, which over expresses Hv1 by RT-PCR analysis (data not shown). FIG. 5B is a chart of data collected in the wound healing assays of FIG. 5A 14 hours after wounding that reports fraction of wound remaining as a function of the concentration of compounds 11 (1-(1,3-benzoxazol-2-yl)guanidine), 7 (1-(1,3-benzoxazol-2-yl)guanidine), and 13 (5-chloro-2-guanidinobenzimidazole).

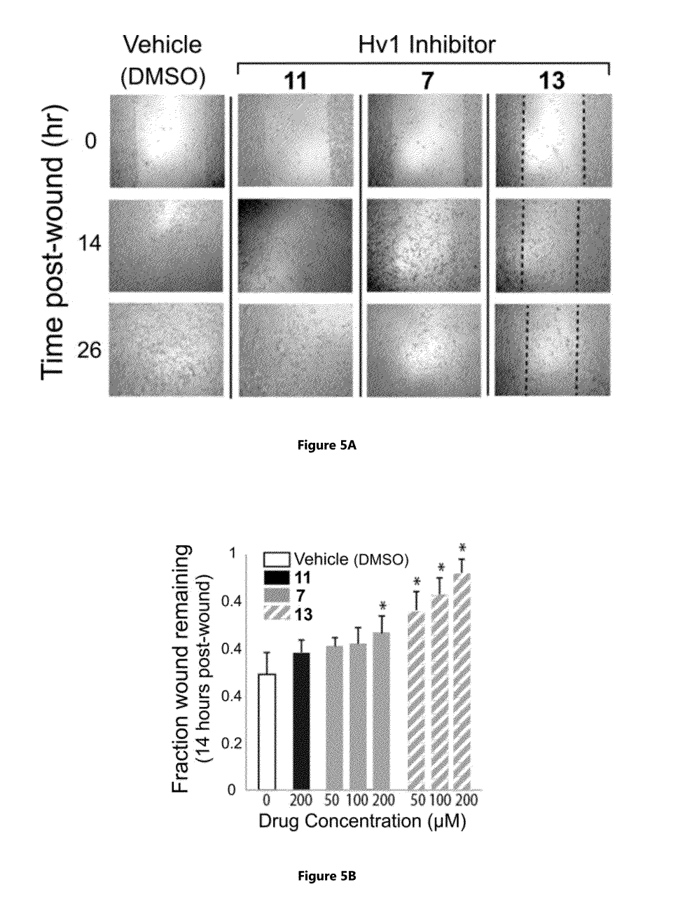

### DETAILED DESCRIPTION OF THE INVENTIONS

Certain embodiments of the present invention provide compounds that comprise at least one guanidine moiety and have a binding affinity for the VSD of a voltage-gated ion channel. In some embodiments, the voltage-gated ion channel is a proton channel, such as Hv1. In some embodiments, compounds according to the invention comprise at least one guanidine moiety in a five-membered aromatic ring. In some embodiments, compounds according to the invention that comprise at least one guanidine moiety have a binding affinity for the VSD in a range selected from millimolar, micromolar, nanomolar, picomolar, and femtomolar. Non-limiting examples of compounds according to the present invention that comprise at least one guanidine moiety and have a VSD binding affinity include those defined by below-illustrated formulas I, II, and III.

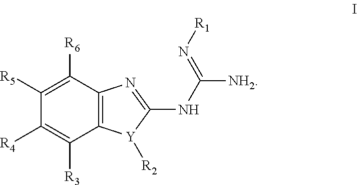

In formula I, Y is either N or S; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is S.

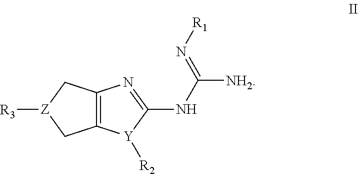

In formula II, Z is either C or N; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, wherein R2 is not present when Y is S.

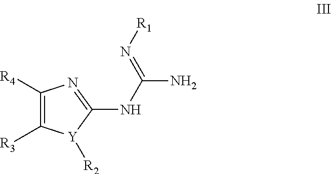

In formula III, Y is either N or S; R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen, and R2 is not present when Y is 0.

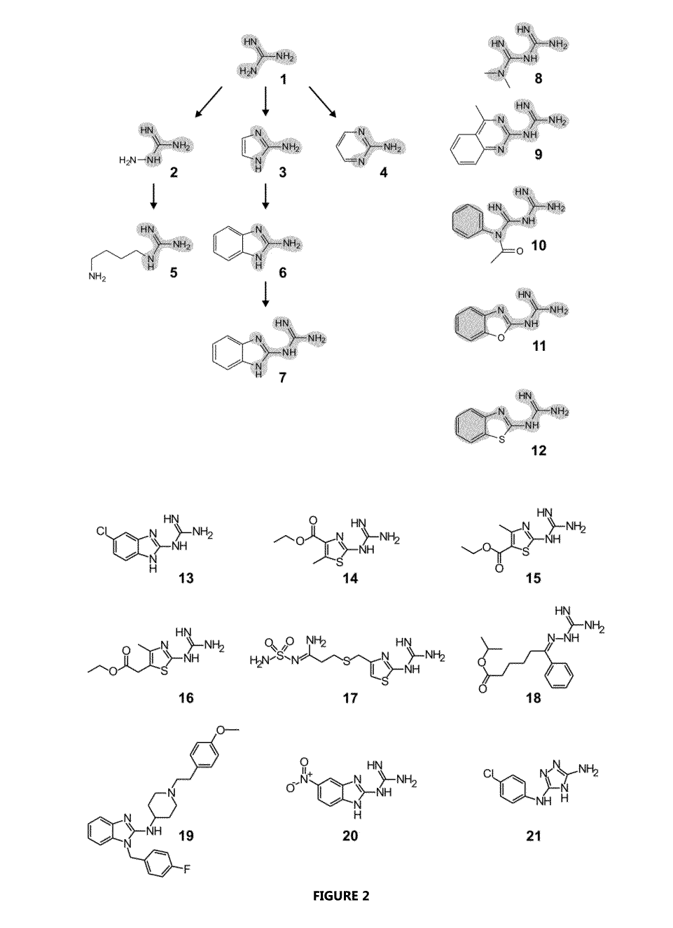

Further non-limiting examples of compounds according to the present invention include those illustrated in FIG. 2.

In some embodiments, compounds according to the present invention that comprise at least one guanidine moiety and have a VSD binding affinity are further characterized by having the property of inhibiting a ion transport activity of a voltage-gated ion channel. In some embodiments, the voltage gated ion channel is a proton channel, such as Hv1, and the transport activity is proton transport. In some embodiments, the strength of such proton transport inhibition is in a range selected from about 10% or more, 20% or more, 30% or more, 40% or more, 50% or more, 60% or more, 70% or more, 80% or more, and 90% or more. In some embodiments, the voltage-gated channel is Hv1.

Some embodiments of the present invention provide methods of inhibiting ion transport by a voltage-gated ion channel in a subject in need thereof by administering to the subject a therapeutically effective amount of a compound according to the present invention. Such a subject may be in need of such methods for a variety reasons, examples of which are affliction with a disease and/or condition associated with the ion transport activity of a voltage-gated ion channel. In some embodiments, the voltage-gated ion channel is a proton channel, such as Hv1, and the ion transport activity is proton transport. A non-limiting example of such a disease is cancer characterized by having cells that overexpress Hv1. A non-limiting example of such a condition is cerebral ischemia involving NOX-mediated neuronal death facilitated by Hv1 proton transport activity. Another non-limiting example of such a condition is tissue damage in chronic inflammation produced by oxidative stress facilitated by Hv1 proton transport activity.

Routes of administering a compound according to the present invention include, without limitation, oral, percutaneous, parental, and intravenous. And administering a therapeutically effective amount of a compound according to the present invention can be accomplished by administering an amount of the compound reasonably calculated to provide ion (e.g., proton) transport inhibiting levels of the compound to intended tissues and/or cells of the subject in need thereof. Exemplary amounts of the compound include amounts that provide to intended tissues and/or cells concentrations of the compound selected from the range consisting of millimolar, micromolar, nanomolar, picomolar, and femtomolar. Additional exemplary amounts of the compound include 10 mg/kg, 5 mg/Kg, 0.5 mg/Kg, 0.05 mg/Kg.

Some embodiments of the present invention provide pharmaceutical formulations that comprise a compound of the present invention together with excipients in solid or liquid dosage forms. Exemplary solid dosage forms include tablets, capsules, pills, and the like. Exemplary liquid dosage forms include injectable solutions, drinkable solutions, and the like.

In some embodiments, one or more compound(s) according to the present invention are coadministered with additional drugs or active agents that have voltage-gated ion channel inhibition activity. Such coadministration can comprise simultaneous administration or sequential administration within a time period selected from a week or less, a day or less, 12 hours or less, six hours or less, four hours or less, one hour less, in 15 minutes or less. Examples of such additional drugs or active agents include zinc, verapamil, 4-aminopyridine, PAP-1, correolide, TRAM-34, azimilide, imipramine, flecainide, and lamotrigine. In some embodiments, one or more compound(s) according to the present invention are coadministered with additional drugs or active agents that have inhibition activity on the NADPH oxidase, such as apocynin, VAS2870, ML171, GKT136901, celastrol.

Table I provides the Hv1 proton transport % inhibition, concentration (μM) for exemplary, non-limiting compounds according to the instant invention and a negative control (creatine).

| | Hv1 proton transport |
|---|---|
| Compound | % inhibition |
| aminoguanidine | 4.3 ± 2.3 |
| | 200 μM |
| 2-aminoimidazole | 21.2 ± 5.1 |
| | 200 μM |
| 2-aminopyrimidine | 2 ± 1 |
| | 200 μM |
| agmatine | 18.5 ± 1.5 |
| | 200 μM |
| 2-aminobenzimidazole | 29.8 ± 2.5 |
| | 200 μM |
| 2-guanidinobenzimidazole | 82.6 ± 2.25 |
| | 200 μM |
| 1,1-dimethylbiguanide | 6.8 ± 2.25 |
| | 200 μM |
| 2-guanidino-4-methylquinazoline | 31.7 ± 0.7 |
| | 200 μM |
| N-(guanidine-imino-methyl)-N- | 0.5 ± 1 |
| phenylacetamide | 200 μM |
| 1-(1,3-benzoxazol-2-yl)guanidine | 1 ± 1 |
| | 200 μM |
| 1-(1,3-benzothiazol-2-yl)guanidine | 81 ± 1.9 |
| | 200 μM |
| 5-chloro-2-guanidinobenzimidazole | 93.6 ± 1.1 |
| | 20 μM |
| ethyl 2-[[amino(imino)methyllamino]-5- | 39.4 ± 3.4 |
| methyl-1,3-thiazole-4-carboxylate | 200 μM |
| ethyl 2-[[amino(imino)methyllamino]-4- | 31 ± 3.4 |
| methyl-1,3-thiazole-5-carboxylate | 200 μM |
| ethyl (2-[[amino(imino)methyl]amino1-4- | 68.2 ± 2.2 |
| methyl-1,3-thiazol-5-yl)acetate, | 200 μM |
| 3-([2-(diaminomethyleneamino)thiazol-4- | 21.5 ± 2.7 |
| yllmethylthio)-N′- | 200 μM |
| sulfamoylpropanimidamide | |
| Isopropyl 6-(guanidinoimino)-6- | 73 ± 3 |
| phenylhexanoate, HNO₃ | 200 μM |
| 1-[(4-fluorophenyl)methyl]-N-[1-[2-(4- | 67 ± 6 |
| methoxyphenyl)ethyl]-4- | 200 μM |
| piperidylibenzoimidazol-2-amine | |
| 5-nitro-2-guanidinobenzimidazole | 57 ± 10 |
| | 4 μM |
| N(3)-(4-chlorophenyl)-4H-1,2,4-triazole-3,5- | 28 ± 7 |
| diamine | 40 μM |
| Creatinine | 0 |
| | 600 μM |

### Example 1

### Channel Expression in Xenopus Oocytes

Constructs containing the sequence of the human Hv1 channel were generated from cDNA provided by David Clapham (Ramsey et al., 2006. A voltage gated proton-selective channel lacking the pore domain. Nature 440, 1213-1216, the content of which is hereby incorporated by reference in its entirety) and from IMAGE clone 5577070 (Open Biosystems). All constructs were subcloned in the pGEMHE vector (Liman et al., 1992) by the SOEing technique. Plasmids were linearized with either NheI or SphI restriction enzymes (New England Biolabs) before in vitro transcription. RNA synthesis was carried out with a T7 mMessage mMachine transcription kit (Ambion). Ci-VSOP was in the pSD64TF expression vector (Krieg and Melton, 1984). The linearized plasmid was transcribed with SP6 RNA polymerase. cRNAs were injected in Xenopus oocytes (50 nl per cell, 0.3-1.5 μg/μl) 1-3 days before the electro-physiological measurements. Cells were kept at 18° C. in ND96 medium containing 96 mM NaCl, 2 mM KCl, 1.8 mM CaCl2, 1 mM MgCl2, 10 mM HEPES, 5 mM pyruvate, 100 μg/ml gentamycin (pH 7.2).

Cultured Cells Expressive Native Hv1.

BV-2 and THP-1 cells were gifts from Heike Wulff (University of California, Davis) and Albert Zlotnik (University of California, Irvine), respectively. RAW264.7 cells were from ATCC (TIB-71). BV-2 and RAW264.7 cells were maintained in Dulbecco's Modified Eagle's Medium supplemented with 10% FBS. THP-1 cells were maintained in RPMI-1640 medium supplemented with 10% FBS and 50 μM 2-mercaptoethanol. Cells were kept at 37° C. in a 5% CO2 incubator.

### Example 2

### Hv1 Inhibitors

All the compounds tested were at the highest purity commercially available. Guanidine hydrochloride was from MP Biomedicals. Aminoguanidine hydrochloride was from Acros orgnanics. 2-aminoimidazole sulfate, 2-aminopyrimi-dine, agmatine sulfate, 2-aminobenzimidazole, 2-guanidinobenzimidazole, 1,1-dimethylbiguanide hydrochloride, 2-guanidino-4-methylquinazoline hydrochloride, and N-(guanidino-imino-methyl)-N-phenylacetamide hydrochloride, were from Sigma-Aldrich. 1-(1,3-benzoxazol-2-yl)guanidine and 1-(1,3-benzothiazol-2-yl)guanidine were from both ChemDiv and Sigma-Aldrich.

The compounds were directly dissolved in the recording solutions at the desired final concentrations or prepared as 100× stock solutions in the same medium. To keep GBOZ in solution at the highest concentrations tested on WT Hv1 channels, DMSO was added to a maximal ratio of 5% v/v for the 4 mM solution. We tested recoding solutions with DMSO up to 10% v/v on inside-out patches containing Hv1 channels, and 5% was the maximal ratio that we were able to use without altering the measured proton currents or compromising the stability of the patch under perfusion.

We estimated the pKa of the guanidinium group of the tested inhibitors using the pKa calculation plugin of Marvin (http://www.ChemAxon.com). With the exception of 2-aminopyrimidine, the compounds were predicted to be primarily in the protonated and positively charged form at pH=6.0. Compounds 7, 11, and 12 were also analyzed as free ligands in PROPKA3.1 (http://propka.ki.ku.dk) (Sondergaard et al., 2011). A charge of +1 was predicted for the three compounds under the pH conditions used for the measurements

### Example 3

### Patch-Clamp Assay

Electrophysiological measurements on oocytes were performed in inside-out and outside-out patch configurations using an Axopatch 200B amplifier controlled by pClamp10 software through an Axon Digidata 1440A (Molecular Devices). The bath solution contained 100 mM 2-(N-morpholino)ethanesulphonic acid (MES), 30 mM tetraethylammonium (TEA) methanesulfonate, 5 mM TEA chloride, 5 mM ethyleneglycol-bis(2-ami-noethyl)-N,N,N0,N0-tetra-acetic acid (EGTA), adjusted to pH 6.0 with TEA hydroxide. For recordings carried out in the absence of pH gradient (pHi=pHo=6.0), the pipette solution had the same composition of the bath solution. Some of the measurements were performed in the presence of a pH gradient (pHi=6.0, pHo=7.5). In these cases the extracellular solution contained 100 mM 4-(2-hydroxyethyl)-1-piperazineethanesulfonic acid (HEPES), 40 mM TEA methanesulfonate, 5 mM TEA chloride, adjusted to pH 7.5 with TEA hydroxide. Native proton currents in BV-2, RAW264.7, and THP-1 were measured in whole-cell configuration with an intracellular solution containing: 90 mM TEA methanesulfonate, 100 mM MES, 2 mM MgCl2, 2 mM EGTA, adjusted to pH 6.0 with TEA hydroxide. The extracellular solution contained: 85 mM TEA methanesulfonate, 100 mM HEPES, 3 mM CaCl2, 1 mM EGTA, adjusted to pH 7.5 with TEA hydroxide. All measurements were performed at 22° C.±2° C. Pipettes had 2-4 M access resistance. Current traces were filtered at 1 kHz, sampled at 5 kHz and analyzed with Clampfit10.2 (Molecular Devices) and Origin8.1 (OriginLab).

### Example 4

### Wound-Healing Assay

The steps of this assay are to grow in six well plates monolayers of cancer cells that express voltage-gated ion channel (e.g., Hv1). The monolayers are mechanically wounded with a 200 μl pipette tip and washed to remove detached cells. Following wounding, cells are maintained in 2 mL of media with 0, 50, 100 or 200 μM of voltage gated proton channel inhibitor test compounds. Images of the wound are taken with an inverted bright field microscope and a digital camera at the time it was created and at 14 and 26 hour time points thereafter. The images are used to determine the distance between the confluent edges of cells lining the wound using software such as NIS-Elements. From this data, the fraction of the original wound remaining is calculated as a function of time, which is a measure of cancer cell proliferation, migration, and/or invasion rates. The fraction of wound remaining was calculated as wound distance at the specified time point divided by initial wound distance. Voltage-gated ion channel inhibitor test compounds that slow the rate of wound healing in this assay are potential chemotherapeutic compounds.

The skilled artisan will recognize the interchangeability of various features from different embodiments. Similarly, the various features and steps of the present invention discussed above, as well as other known equivalents for each such feature or step, can be mixed and matched by one of ordinary skill in this art to obtain compounds and methods of the invention described herein. Although the disclosure has been provided in the context of certain embodiments and examples, it will be understood by those skilled in the art that the disclosure extends beyond the specifically described VSD comprising voltage-gated proton channel embodiments to other alternative embodiments, such as VSD comprising voltage-gated sodium, potassium, and calcium channels and/or uses and obvious modifications and equivalents thereof. Accordingly, the disclosure is not intended to be limited by the specific disclosures of embodiments herein.

What is claimed is:

1. A method of inhibiting a proton transport activity of a human voltage-gated proton channel (Hv1 channel) in a vertebrate cell that expresses said Hv1 channel, the method comprising contacting said vertebrate cell with an amount of a compound sufficient to inhibit said proton transport activity, wherein said compound comprises a chemical structure according to formula I: 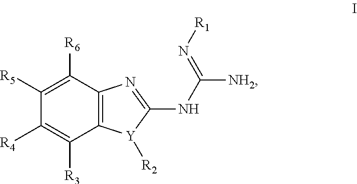
and
wherein:
Y is either N or 5;
R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen;
R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen;
R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and
R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen,
wherein R2 is not present when Y is S.

2. The method of claim 1, wherein said compound comprises at least one member selected from the group consisting of a 2-aminobenzimidazole, a 2-guanidinobenzimidazole, a 2-guanidino-4-methylquinazoline, 1-(1,3-ben-zothiazol-2-yl)guanidine, and a 5-chloro-2-guanidinobenzimidazole.

3. The method of claim 1, wherein said compound comprises at least one member selected from the group consisting of a 2-guanidinobenzimidazole, a 1-(1,3-ben-zothiazol-2-yl)guanidine, and a 5-chloro-2-guanidinobenzimidazole.

4. The method of claim 1, wherein said compound comprises 2-guanidinobenzimidazole.

5. The method of claim 1, wherein said compound comprises 1-(1,3-ben-zothiazol-2-yl)guanidine.

6. The method of claim 1, wherein said compound comprises 5-chloro-2-guanidinobenzimidazole.

7. A method of inhibiting proton transport by a Hv1 channel in a vertebrate cell that expresses said Hv1 channel, the method comprising contacting said vertebrate cell with an amount of a compound sufficient to inhibit said proton transport, wherein said compound comprises a chemical structure according to formula II: 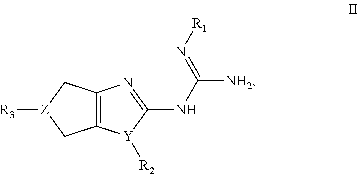
and
wherein:
Z is C or N;
R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen;
R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and
R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen,
wherein R2 not present H when Y is S.

8. A method of inhibiting proton transport by Hv1 channel in a vertebrate cell that expresses said Hv1 channel, the method comprising contacting said vertebrate cell with an amount of a compound sufficient to inhibit said proton transport, wherein said compound comprises a chemical structure according to formula III: 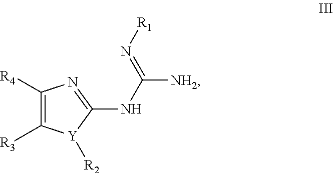
and
wherein:
Y is either N or 5;
R1 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen;
R2 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen;
R3 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen;
R4 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen;
R5 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen; and
R6 is selected from the group consisting of an H, a CH₃, an OH, a NH₂, a NO₂, a SO₃H, a COOH, a COCH₃, a CONH₂, a CF₃, a saturated alkyl, an unsaturated alkyl, an aryl, and a halogen,
wherein R2 is not present when Y is S.
